(19) United States
(12) Patent Application Publication
(10) Pub. No.: US 2010/0298718 A1
(43) Pub. Date: Nov. 25, 2010

(54) Multiple Mode, Portable Patient Monitoring System

(76) Inventors: Jeffrey Jay Gilham, Sammamish, WA (US); Mernilo D. Cruz, JR., Issaquah, WA (US); Patricia Ruth Walters, Issaquah, WA (US)

Correspondence Address: PATENTMETRIX, 14252 CULVER DR. BOX 914, IRVINE, CA, 92604, US

(21) Appl. No.: 12/768,714

(22) Filed: Apr. 27, 2010

Related U.S. Application Data
(60) Provisional application No. 61/173,167, filed on Apr. 27, 2009.

(51) Int. Cl. A61B 5/0205

(52) U.S. Cl. 600/484

(57) ABSTRACT

The specification discloses a number of embodiments directed to patient monitoring systems. The system determines a physiological parameter of a patient and includes an input device for receiving a plurality of physical inputs from a user, such as finger taps, and communicating to a memory interval data indicative of the frequency at which the plurality of physical inputs are received from the user within a time period and a processor in data communication with said memory wherein said processor determines at least one physiological parameter of a patient based upon said interval data.

### CROSS-REFERENCE TO RELATED APPLICATIONS

The present application relies on U.S. Provisional No. 61/173,167, filed on Apr. 27, 2009, for priority.

### FIELD OF THE INVENTION

The present invention relates generally to the field of patient monitoring systems. In particular, the present invention relates to a portable system for monitoring patient parameters that can be used for both spot-check monitoring and continuous monitoring, and that can communicate with a patient database to receive and transfer data.

### BACKGROUND OF THE INVENTION

Patient monitoring systems are commonly used in hospitals, such as in intensive care units (ICUs), for monitoring patient status and condition. Conventional patient monitoring systems typically include a bedside or portable monitor having one or more sensors attached to the patient, for sensing parameters such as ECG, blood pressure, blood oxygen, blood glucose and temperature. The output from the sensors is sent to a system processor, which subsequently processes the measured data. These values may then be displayed on a video display screen or stored for later analysis. Data representing the measured physiological parameters is typically displayed as waveforms and/or numerical values.

Conventional patient monitoring systems are also capable of handling critical patient events or alarm conditions. For example, when the value of one of the physiological parameters being monitored exceeds a predetermined threshold value and/or meets predetermined alarm criteria, an alarm is activated by the bedside monitor and subsequently transmitted to a central monitoring station. The alarm can be annunciated at the central station in various ways, such as by highlighting relevant parameter information. An audible alarm is also typically generated at the central station.

Prior art patient monitoring systems also include systems that provide output signals indicative of normal, above normal or below normal sensed conditions. The signals may be used to monitor a condition and may be combined so that specific combinations of abnormal signals provide an indication of the condition of the patient.

Most of the conventional patient monitoring devices operate in only one mode, typically either spot-check mode or continuous patient monitoring. In spot-check mode, patient monitoring systems support collection of basic vitals for multiple patients, on demand. In continuous monitoring mode, a patient is continuously connected to a monitoring system and vitals are recorded. Monitors that are dedicated to working in continuous mode are not capable of handling data from multiple patients simultaneously.

In addition, primary users of typical patient monitoring systems include the clinical staff responsible for patient care in general care wards, medical intensive care units (MICU), surgical intensive care units (SICU), emergency departments, triage, outpatient surgery facilities and urgent care centers. Most of the existing patient monitoring systems however tend to be cumbersome and do not allow for convenient information sharing amongst such users or for convenient information capture and inputting of data.

There is therefore a need for a patient monitor that will support and complement the work flow of its users and contribute to a significant reduction in the time required to acquire and document vital sign data. Thus, what is needed is a patient monitoring system that is not only capable of receiving data from and transferring data to a central patient database, but also provides the physicians or clinicians with efficient and effective means for quickly analyzing data in an information-rich environment.

Also needed is a patient monitor that is flexible such that it can readily adapt to the changing acuity levels in patient care environments. There is further a need for a patient monitoring device that is portable, lightweight, and can operate in multiple modes.

### SUMMARY OF THE INVENTION

The present specification discloses a system for determining a physiological parameter of a patient, the system comprising: an input device for receiving a plurality of physical inputs from a user and communicating to a memory data indicative of the frequency at which said plurality of physical inputs are received from the user within a time period; and a processor in data communication with said memory wherein said processor determines the value of a physiological parameter based upon said data. Data collected by the device represents the time of each physical input by said user. The input is preferably in the form of discrete, binary inputs, as opposed to waveform or non-discrete inputs.

Optionally, the physiological parameter is at least one of pulse rate or respiratory rate. The input device is a touch screen display. The plurality of physical inputs received from the user are finger-taps on said touch screen display. The input device is at least one of a touch screen display, mouse pad, or keyboard. The plurality of physical inputs received from the user are finger-taps on at least one of said touch screen display, mouse pad, or keyboard. The time period is fixed. The fixed time period is system-defined. The fixed time period is user-defined. The time period is variable and is dynamically determined by the system when the user starts and stops the physical input. The processor determines the value of the physiological parameter by averaging all of interval data generated by said physical inputs. The average interval data is derived by calculating a straight arithmetic average of the measured intervals. The average interval data is derived by rejecting the longest and shortest interval data generated by the physical inputs and then averaging the remaining data. The average interval data is derived by calculating a median value of the physical inputs. The average interval data is derived by analyzing clusters of intervals and using a clustering algorithm to reject long or short intervals that should not be included. The average interval data is displayed in real time and continuously updated with each physical input.

In another embodiment, the system for determining a physiological parameter of a patient comprises an input device for receiving a plurality of physical inputs from a user, wherein said physical inputs comprise finger taps, and communicating to a memory data indicative of the frequency at which said plurality of physical inputs are received from the user within a time period, wherein said data comprises interval data; and a processor in data communication with said memory wherein said processor determines at least one of the patient's respiration rate or pulse rate based upon said data. The processor determines the value of the physiological parameter by averaging all of interval data generated by said physical inputs.

These, and other embodiments, will become better understood in the detailed description as read in light of the accompanying figures.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1A is a block diagram of an exemplary communications interface between a patient monitoring system, as in the present invention and a centralized patient database;

FIG. 1B is a block diagram of an exemplary communications interface between a patient monitoring system, as in the present invention and a centralized patient database;

FIG. 2A illustrates a perspective view of one embodiment of the portable patient monitoring system of the present invention;

FIG. 2B illustrates a perspective view of one embodiment of the portable patient monitoring system of the present invention;

FIG. 2C shows an expanded view of one embodiment of the portable patient monitoring system of the present invention;

FIG. 3A represents an illustrative view of a general exemplary layout of a GUI in Spot Check mode;

FIG. 3B represents an illustrative view of a general exemplary layout of a GUI in Continuous Monitoring mode;

FIG. 3C illustrates a plurality of device status icons, as used within a GUI of the portable patient monitoring system of the present invention;

FIG. 3D illustrates a plurality of monitor function keys, as used within a GUI of the portable patient monitoring system of the present invention;

FIG. 3E illustrates details of the icons in the NIBP zone, as employed in a GUI of the present invention;

FIG. 3F illustrates details of the icons in the respiration measurement area, as employed in Spot Check mode;

FIG. 3G illustrates details of the icons in the Temperature zone, as employed in a GUI of the present invention;

FIG. 3H illustrates details of the icons in the SPO₂ zone, as employed in a GUI of the present invention;

FIG. 4A illustrates a general exemplary layout of a GUI in Spot Check mode;

FIG. 4B is an illustration of a general exemplary layout of a GUI in Continuous Monitoring mode;

FIG. 5 shows an exemplary application window for Spot Check mode;

FIG. 6 shows an exemplary application window for Continuous Monitoring mode;

FIG. 7 shows an exemplary GUI window when the patient monitoring system of the present invention is operated in a NIBP Protocol mode; and

FIG. 8 depicts an exemplary flowchart for using one embodiment of the tap pad of the present invention.

### DETAILED DESCRIPTION OF THE INVENTION

The present invention is directed towards a patient monitoring system, and in particular, a multiple mode patient monitoring system. In one embodiment, the system determines a physiological parameter of a patient, the system comprising an input device for receiving a plurality of physical inputs from a user and communicating to a memory data indicative of a frequency of how often said plurality physical inputs are received from the user within a time period; and a processor in data communication with said memory wherein said processor determines the physiological parameter based upon said data.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

As further discussed below, the present specification discloses a number of different portable patient monitoring systems. One of ordinary skill in the art would appreciate that each embodiment described herein comprises a plurality of programmatic modules that have been designed, written, and/or configured to perform the described functionality when executed by a processor. Each monitoring system embodiment comprises at least one processor in data communication with a memory and executing a plurality of programmatic modules which, when executed, cause the described functions, actions, data flows, or results to be performed or generated. Such data is then communicated to one or more hardware displays, including CRT monitors, touch screens, LCD screens, flat panel displays, or any other display known to persons of ordinary skill in the art.

In one embodiment, the patient monitoring system of the present invention can be operated in “Spot-Check” Mode. In another embodiment, the patient monitoring system of the present invention can be operated in “Continuous Monitoring Mode”. In addition, the present invention is directed towards a patient monitoring system that is portable. And, more particularly, the present invention is directed towards a patient monitoring system that is portable and can be operated in multiple modes, namely “Spot Check” and “Continuous Monitoring”.

In one embodiment, the patient monitoring system of the present invention supports both adult and pediatric patients and is configurable as a function of patient type. In one embodiment, the selection of a patient type results in automatic configuration of alarm limits and operational settings (such as initial inflation pressures, maximum inflation pressures) based on pre-defined default settings. In other embodiments, automatic configuration is provided as a function of the device type; operating mode (such as continuous or spot check); specific caregiver (such as various type of specialists and/or an actual caregiver authorized to use the device), and specific care area (such as, but not limited to, ICU, SICU, NICU, PICU, or emergency/trauma center). Such automatic configuration includes all configurable features of the data analysis and presentation such as, but not limited to, alarm settings, user preferences for screen layout including font color, size, etc, alarm tone selection, order of parameters on display, etc.

For example, in one embodiment, a pre-set profile is created, stored, and available for access by a caregiver based upon patient type. In one configuration, patient type can be defined in terms of age (adult or pediatric). If “adult” is chosen, a first set of values or modes for each of the following variables is selected, stored, and available for access when a caregiver chooses the “adult” profile from the patient monitoring system menu: blood pressure cuff initial inflation pressure of 110, 160 or 210 mmHg; alarm thresholds for SpO₂ at values less than 92 and greater than 100; alarm thresholds for patient temperature at values less than 97° F. and greater than 102.0° F.; alarm thresholds for systolic pressure at values less than 100 mmHg and greater than 180 mmHg; alarm thresholds for diastolic pressure at values less than 45 mmHg and greater than 110 mmHg; alarm thresholds for mean arterial pressure at values less than 70 mmHg and greater than 120 mmHg; and alarm thresholds for pulse rate at values less than 50 bpm and greater than 140 bpm. Further, SpO2 pulse tones can be set to on or off, depending upon patient need. Still further, NIBP protocol mode can be set to on or off; if set to on, an NIBP mode may be selected from the menu and includes Interval, Stat, Proto1, Proto2, or Proto3, as described below. Still further, if Interval Mode is selected, the NIBP readings are taken at 5, 10, 15, 30, 60, or 120 minute intervals. Still further, if Protocol mode is selected, the measurements are taken at user-defined intervals.

In addition, in adult mode, the user can set to which location, care area, or caregiver an alarm should be routed, among other variables, such as user preferences for screen layout including font color and size, alarm tone selection, and order of parameters on display.

If “pediatric” is chosen, a second set of values or modes for each of the following variables is selected, stored, and available for access when a caregiver chooses the “pediatric” profile from the patient monitoring system menu: blood pressure cuff initial inflation pressure of 110, 160 or 210 mmHg; alarm thresholds for SpO₂ at values less than 92 and greater than 100; alarm thresholds for patient temperature at values less than 97° F. and greater than 102.0° F.; alarm thresholds for systolic pressure at values less than 80 mmHg and greater than 140 mmHg;

alarm thresholds for diastolic pressure at values less than 40 mmHg and greater than 90 mmHg; alarm thresholds for mean arterial pressure at values less than 50 mmHg and greater than 100 mmHg; and alarm thresholds for pulse rate at values less than 70 bpm and greater than 140 bpm. Further, SpO2 pulse tones can be set to on or off, depending upon patient need. Still further, NIBP protocol mode can be set to on or off; if set to on, an NIBP mode may be selected from the menu and includes Interval, Stat, Proto1, Proto2, or Proto3, as described below. Still further, if Interval Mode is selected, the NIBP readings are taken at 5, 10, 15, 30, 60, or 120 minute intervals. Still further, if Protocol mode is selected, the measurements are taken at user-defined intervals.

In addition, in pediatric mode, the user can set to which location, care area, or caregiver an alarm should be routed, among other variables, such as user preferences for screen layout including font color and size, alarm tone selection, and order of parameters on display.

In one embodiment, a pre-set profile is created, stored, and available for access by a caregiver based upon operating mode. Operating mode can be defined in terms of continuous or spot check.

In another embodiment, a pre-set profile is created, stored, and available for access by a caregiver based upon the caregiver type (type of specialist or identity of authorized personnel) or care area (such as, but not limited to, ICU, SICU, NICU, PICU, or emergency/trauma center). If a particular specialist is chosen, a set of values specific to that medical specialty is chosen to be prominently displayed, such as NIBP, systolic pressure, diastolic pressure, and mean pressure for cardiologists, while other variables are not displayed or minimized from view. If a profile specific to the identity of a particular caregiver is chosen, a set of values specific to the needs and preferences of that person is chosen, including user preferences for screen layout including font color and size, alarm tone selection, order of parameters on display, type of data displayed, among other variables. If a profile specific to a particular care area is chosen, a set of values specific to the needs and preferences of that care area is chosen, including user preferences for screen layout including font color and size, alarm tone selection, order of parameters on display, type of data displayed, among other variables.

In yet another embodiment, a pre-set profile is created, stored, and available for access by a caregiver based upon the patient's disease or ailment. If a particular ailment or indication is chosen, a set of values specific to that medical ailment is chosen to be prominently displayed, such as NIBP, systolic pressure, diastolic pressure, and mean pressure for heart disease.

One of ordinary skill in the art would appreciate that, for each of the above described profiles, selection of a particular pre-set profile would automatically cause a controller within the patient monitoring system to configure operations, including sensor operation, peripheral operation, blood pressure cuff operation, and alarm operation, in accordance with the stored pre-set values.

In one embodiment, the patient monitoring system of the present invention is a pre-configured, portable monitor with capabilities for receiving measurements for, or data indicative of, physiological parameters of a patient, including, but not limited to Non-Invasive Blood Pressure (NIBP), Pulse Oximetry (SpO₂) and Temperature. The patient monitor of the present invention is capable of receiving measurements for, or data indicative of, and displaying, systolic pressure, diastolic pressure, mean pressure and pulse rate, which are described in greater detail below.

In one embodiment, initiation of an NIBP reading while NIBP is in an alarm state, results in the alarm being silenced and the NIBP reading completing. The monitor is capable of supporting multiple, user-adjustable initial inflation pressure settings. In one embodiment, NIBP readings may be taken in one of (4) selectable measurement modes: SINGLE, STAT, AUTO and PROTOCOL, which are described below in greater detail.

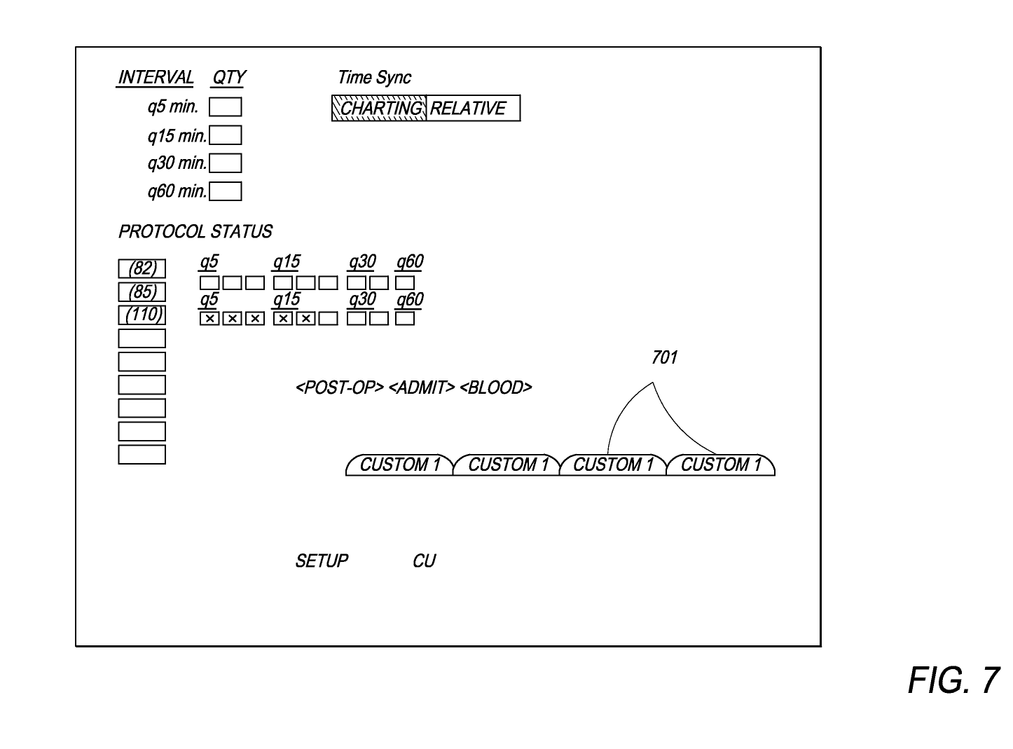

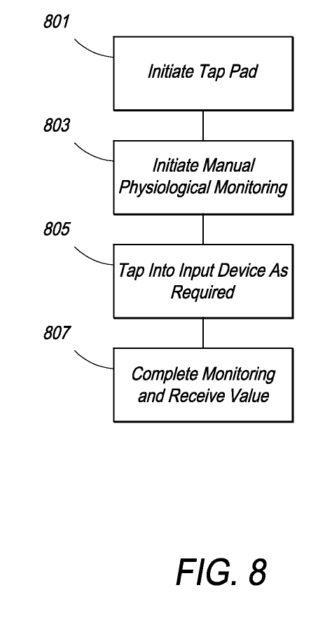

When the user initiates an NIBP monitoring event while the measurement mode is set to operate in PROTOCOL mode, the patient monitoring system acquires a plurality of user-defined readings at a user-defined time interval over multiple, sequential, user-defined time durations. For example, a user may specify the monitor to take 5 readings at every 5 minutes, then 5 readings at every 10 minutes, and so on. In addition, NIBP protocol mode allows the user to create a custom, variable schedule for taking automatic NIBP readings, described in greater detail below with respect to FIG. 7.

During PROTOCOL mode of operation, the monitor provides a visual indication of status, including the number of completed readings for each specified interval. During PROTOCOL operational mode, on completion of an NIBP reading, the monitor notates, as part of the patient record, all physiologic measurement values, such as SpO₂, pulse rate, and temperature, registered at the completion time of the NIBP reading. Also during PROTOCOL operation, in one embodiment, if a manual reading is initiated and completes within 30 seconds of the next scheduled reading, the monitor counts the manual reading as the next scheduled measurement. The monitor further provides a count-down timer, displayed in minutes and seconds, to indicate the time remaining before the next auto reading occurs. When initiating PROTOCOL operation, the initial measurement begins immediately and subsequent measurements then follow in a format that charts measured values against time.

When the operational mode is set to SINGLE, the patient monitoring system of the present invention, upon user initiation, acquires, stores, and displays one single NIBP measurement when activated.

In STAT measurement mode, the patient monitoring system, upon user initiation, acquires multiple sequential readings performed over a pre-defined duration of time, such as 5 minutes. That is, the device records as many readings as possible, or a pre-set number of readings, within the specified duration.

When operation is set to AUTO, the patient monitoring system of the present invention, upon user initiation of NIBP monitoring, results in the automatic acquisition of NIBP readings performed at a specified time intervals, such as every 30 minutes. The readings may continue indefinitely or until manually stopped. With the exception of SINGLE, all NIBP measurements are charted relative to time, which typically, although not necessarily, initiates the readings at a rounded time period of five-minute increments (i.e. 10:05, 10:15, etc). The monitor supports SINGLE, STAT, AUTO and PROTOCOL measurement modes when the monitor's operating mode is set to MONITORING. Subsequent inflation pressures are performed as a function of the previous measurement.

In the event an NIBP measurement cannot be completed successfully on first try, the monitor is capable of providing one additional measurement attempt. In the event an NIBP measurement cannot be completed successfully following one additional measurement attempt, the patient monitor provides an annotation to the NIBP section of the measurement record to denote failure to acquire a reading and/or generates an error signal.

The patient monitor supports the capability for the user to manually enter supplemental information into the measurement record as described below. The user can also specify certain measurement modifiers such as, but not limited to measurement location (i.e. right arm, thigh, etc.) and patient position (i.e. sitting, supine, etc.). One of ordinary skill in the art would appreciate that data may be received from, and input by, a user through a plurality of means, including keyboard, mouse, touch screen, wireless communication with a handheld device, voice recognition, or any other input mechanism. Once input, the data is stored in relation to the associated variables within local memory and used by the display system controller and/or processor to effectuate the functions described herein.

The patient monitor of the present invention is also capable of measuring and displaying oxygen saturation (SpO₂), pulse rate, plethysmograph waveform and signal quality indicator. The patient monitor of the present invention is also capable of trending oxygen saturation levels, pulse rates and signal quality indicators. The patient monitor of the present invention is capable of supporting any SpO₂ sensor or measurement technologies known to those of ordinary skill in the art.

The patient monitor of the present invention can support SpO₂ monitoring when SpO₂ and NIBP measurements are taken from the same extremity (i.e. SpO₂ finger sensor on the right index finger and NIBP cuff on the right arm). The monitor is also capable of generating an audible pulse tone, emitted concurrently with each pulse beat detected. The pulse tone may change with the level of SpO₂ saturation.

The patient monitor of the present invention is capable of receiving measurements for, or data indicative of, and displaying an episodic (including a single) or continuous temperature reading. When operation is set to “continuous”, the monitor provides continuous measurement and display of the temperature value and supports local alarm management. The patient monitoring system of the present invention allows for the clinician to configure which temperature measurement algorithm is used. In one embodiment, the patient monitoring system of the present invention is capable of acquiring a temperature reading via placement of a temperature probe in an ORAL or AXILLARY measurement site. If a rectal temperature is obtained manually, the clinician is able to manually enter the measurement value into the monitor via the corresponding manual entry fields for temperature value and measurement site.

The monitor provides and displays an episodic temperature measurement for a user-defined period of five (5) minutes or less following measurement completion. When the defined period of time following measurement completion is reached, the temperature parameter zone reverts to a blank state, displaying no value, and the user can only access the reading through the monitor's “Trends” view. If a subsequent temperature measurement is initiated within five (5) minutes of the previously completed measurement, the status and measurement information relative to the measurement reading being attempted is updated in the temperature zone display. The monitor is capable of displaying on-screen status notices/prompts for such things as: sensor/probe warming Up (i.e. displayed until probe reaches 34° C./93° F.); insert probe; reading in progress; reading complete (i.e. value displayed and completion tone emitted); and probe failure/error.

When configured for Spot-Check mode, the patient monitoring system of the present invention can only acquire temperature readings episodically. When configured for Monitoring mode, the patient monitoring system of the present invention can acquire readings either continuously or episodically.

The monitor is capable of displaying pulse rate as measured by NIBP or SpO₂. The monitor is capable of auto-displaying pulse rate from a secondary source (i.e. NIBP) if the primary rate source (i.e. SpO₂) is unavailable. In one embodiment, the monitor can display the following data elements in column format: Time, NIBP (Systolic/Diastolic), NIBP (Mean), Pulse Rate, SpO₂, Temperature, and Status (such as Unverified, Sent/Verified, Deleted).

In one embodiment, as mentioned above, the patient monitoring system of the present invention can be operated in Spot Check Mode. In Spot-Check mode, the monitor functions as an adjunct to the clinical staff for collection and documentation of vital sign data on multiple patients. In spot-check mode, the user can manually perform a single set of physiologic measurements. In addition, the user can associate each measurement set to a specific patient record. Further, in one embodiment, the patient monitoring system of the present invention is capable of collecting a caregiver identifier to document assignment of physiologic measurement sets to specific patient records. Still further, in one embodiment, the monitor is capable of accessing patient identification to facilitate association of physiologic measurement sets for that patient.

In one embodiment, the patient monitoring system can be customized to specific care protocols. Thus, a specific protocol builder within the application can be used to remind the caregiver of particular time-based measurements or other actions that may need to be taken, such as a particular set of readings at particular time intervals.

In another embodiment, the patient monitoring system of the present invention is capable of maintaining multiple sets of measurement records per patient for multiple patients. Further, the system will perform an error check on patient records to ensure the validity of the data.

In one embodiment, following completion of a measurement set, physiologic values will remain displayed on-screen for a period of five minutes, after which all values are removed, stored and viewable only in trends. In one embodiment, stored measurement sets are displayed in a scrollable list identified and sortable by patient name and/or medical record number (MRN).

In one embodiment, when the patient monitoring system of the present invention is operating in Spot Check mode, physiologic alarms are not active, but measured values that exceed the defined high/low limits shall be flagged to distinguish the value(s) as being out of range.

In one embodiment, the patient monitor of the present invention supports manual entry of physiologic reading and associated data not automatically measured by the monitor, such as respiration rate or a temperature value with measurement source of tympanic, which is described in further detail below. In one embodiment, manual entry is facilitated by use of a “graphical mechanism”, such as but not limited to, an active touch pad, for helping automate counting of additional parameters that may possibly be manually measured against a time scale, such as respiration rate (RR) or pulse rate (PR), which is usually depicted as breaths per minute (BPM).

In one embodiment, the patient monitoring system is capable of differentiating between physiologic measurements acquired via the monitor and those manually entered in by the user. In one embodiment, the manually entered values are displayed differently and annotated in code, highlighted, or have a different font. In another embodiment, the patient monitor is capable of supporting manually-entered annotations to stored measurements.

As described earlier, in one embodiment, the patient monitoring system can be customized to care area, specialty, ailment, patient type, care giver, or other user-defined profiles, as referred to as Monitor Profiles. These Monitor Profiles may be created and stored and loaded using the patient monitor of the present invention. Upon selection of a Monitor Profile, default configuration settings are loaded onto the patient monitor. Further, as described above, such automatic configuration includes all configurable features of the data analysis and presentation including alarm settings and user preferences for screen layout.

In one embodiment, to ensure property security of patient data information, the patient monitor of the present invention may require password-protection to control access to back-end administrative settings, defaults, and the like. Further the patient monitor of the present invention may require each caregiver to input unique credentials for associating that user's identification with the authorized transmission of collected patient data to clinical information systems. Further, in one embodiment, the patient monitoring system of the present invention is fully HIPAA compliant.

For example, in one embodiment, the user can configure and customize which elements of a patient's identity are displayed on screen (such as, but not limited to, MRN, initials, etc.). Further, the patient monitor of the present invention can maintain and store an access control list that includes user names, authorization levels and passwords for use in verifying the user's identity and rights. In addition, the system includes an administrative function that provides for maintenance of user access privileges as well as audits of each user's access history. This mechanism ensures authorized access for the clinicians as well as privacy for the patients.

For example, the display system memory may comprise a data structure, such as a table or database, or may comprise programmatic code capable of accessing a remotely stored data structure, such as a table or database, over a network. Within that data structure, patient records may be provided which relates specific patients, as identified using name, social security number, and/or medical record, with a plurality of other variables, including: caregiver type, caregiver identity, ailment type, medical history (including historical data readings of values as described above) and/or Monitor Profile type. In one embodiment, only a caregiver, who has been prompted for, and has input, his or her password, login, RFID tag, or other unique credentials into the system and who is associated with a patient in the patient records, can access the data associated with the patient in the patient record or configure the display according to Monitor Profile specific to the patient. In one embodiment, only caregivers from a specific care area, who have been prompted for, and have input, their password, login, RFID tag, or other unique credentials into the system and who are assigned to the care area that is associated with a patient in the patient records, can access the data associated with the patient in the patient record or configure the display according to Monitor Profile specific to the patient. In one embodiment, the Monitor Profile customizes the extent of patient identity information displayed on screen, including limiting the display to a patient's name, patient's initials, or patient's medical record number or some portion thereof.

In one embodiment, the present invention is directed towards a patient monitoring system that is capable of receiving data from and transferring data to a central patient database. In one embodiment, the patient monitoring system of the present invention supports both inbound and outbound HL7 interfaces to allow for import of ADT information as well as export of patient data, including demographics, to clinical applications, HIS/CIS, etc. Further, the monitor is capable of inbound and outbound interface to the ICS G2 Monitor Loader to support acquisition and viewing of patient data from all ICS G2 applications, including Smart Disclosure, Custom Trends, Bedside View and Clinical Event Interface (CEI).

In one embodiment, a device can access historical data (patient records) from the central database to alert the caregiver of a change in state. For example, the BP of a female patient from nursing home care is usually 140/90. This figure is known to the device, since the history of the patient's BP measurements is loaded into the database, with the readings being sent from the device to the database periodically. The device may therefore also have mean or average values of physiological parameters of the patient for reference. Thus, when the device records a new BP reading for the female patient, and finds her pressure at 110/45, for example, the device can conclude that this is an atypical reading. In such case, the device can alert the caregiver immediately. Alternatively, in case of atypical readings, the device can be configured to start operating automatically in STAT measurement mode as described above, so that the patient monitoring system acquires multiple sequential readings over the next few minutes to makes sure that the measurement is not erroneous and it is indeed an alarm situation and not just an anomaly.

Further, in alerting a caregiver of a change of state for a particular patient, a patient specific alarm can be generated. For example if a patient has high blood pressure, with that patient's blood pressure being around 150/100, on average, and the condition is chronic, then if the patient's systolic pressure rises above 175 (or any other value specified by the patient's caregiver), the patient's caregiver is notified. In one embodiment, the notification may be in the form of a text message on the caregiver's mobile phone. In another embodiment, details of multiple readings (such as in STAT mode) taken when the patient's parameter are not normal, are emailed to the patient's primary caregiver. In this manner, with the availability of patient's histories at points-of-care, the device may be configured to generate “custom alarms”. That is, alarms are customized according to a specific patient's history.

In one embodiment, the patient monitoring system of the present invention is correlated with a patient, where a patient is identified using patient demographics, such as, age, gender, genetic profile and area of residence. Patient demographics may be entered into the patient monitoring system of the present invention using a variety of methods, including inbound ADT interface of patient demographics; barcode scan; touch-screen keyboard entry; or physical keyboard entry.

In one embodiment, the patient monitoring system of the present invention is designed such that it can operate on both wired and wireless networks. Once a patient is identified, associated patient data can be transferred to the system of the present invention via a network. Thus, if the patient ID is sent over the network to a database and an existing patient record is found, the database sends the rest of the patient's demographic data back to the monitor and the user.

In wireless mode, the monitoring system of the present invention may communicate using any wireless application protocol such as, but not limited to IEEE 802.11, Bluetooth, IrDA Home RF, etc. In one embodiment, the monitoring system operates with IEEE 802.11a/g, and supports a suitable protocol such as 802.1x and WPA/WPA2 for security authentication in wireless mode. In one embodiment, the patient monitor also supports TCP/IP addressing and assignment of the monitor's TCP/IP network address from a networked Dynamic Host Communication Protocol (DHCP) server.

If an internet connection is available at the time of data acquisition, a barcode scan or manual entry of the patient identifier is used to query the patient database for a known Patient ID. The patient is either confirmed, where additional patient information such as name and date of birth (DOB) are returned for display during acquisition, or the clinician is prompted to retry or “admit” the patient (manual entry of patient information, described below).

Thus, in operation of one embodiment, when an internet connection is available, the clinician logs into the device by using a username and password, which is verified by the device interface. In one embodiment, the caregiver ID is accepted and authenticated automatically by the device. In one embodiment, the device receives the caregiver's ID by means of an RFID tag worn by the caregiver. In another embodiment, the device is equipped with a biometric reader, which authenticates a caregiver through one of or a combination of biometrics, such as fingerprints, facial recognition or iris of the eye. After authentication, the clinician then enters a patient identifier, such as medical record number, name, or social security number. In one embodiment, the patient ID is entered by scanning a wristband worn by the clinician using a barcode scanner connected with the device. The patient ID is then used to look up patient demographics in the database. If the patient ID is found, then data such as patient name, DOB, and other requested database values (such as height, weight, gender, etc.) are returned. If the patient ID is not found in the database, then the clinician is prompted to retry, cancel, or confirm (for a new patient) the patient ID. If the patient ID is confirmed, then the clinician is prompted to complete required information for admitting patients to the system (name, DOB, etc.). Resultant parameter data is transferred to the database as described in greater detail below. In one embodiment, data transferred to the database also includes the physical location of measurement, such as the ER room or the ICU. As mentioned earlier, location may be used to automatically determine device configuration and settings. In one embodiment, the location may be derived from GPS or wireless connectivity (IP address) of the device.

In another embodiment, if no network connection can be established, the user can manually enter patient demographic data and record readings for subsequent transmission. In one embodiment, the user can enter in patient demographic data such as medical record number, name (first, last, middle initial), height, weight, date of birth (DOB), bed number assigned, etc. In one embodiment, if both height and weight are entered, the monitor will automatically calculate and display body mass index (BMI), and BMI shall be included as part of the exportable patient record. In one embodiment, the patient monitoring system of the present invention provides capability for the user to review and correct patient demographic data.

Thus, in operation, if no wireless or Ethernet connection is available at the time of data acquisition one or more measurements are taken, collected locally in the device, and transferred upon connection. A clinician username is entered, but is not verified by the interface. The clinician then manually enters the patient ID. The clinician takes measurements which are collected and stored in the device until connection to a network. Upon connection to the network, data is sent to the server, including, but not limited to time stamp, user ID, patient ID, device ID, measurement name (x), measurement value (y), and measurement units (z), for all measurements obtained. If a patient ID is recognized, the data collected by the system of the present invention will be appended to the existing patient data in the database. If the patient ID is not recognized, then a new record in the database will be created.

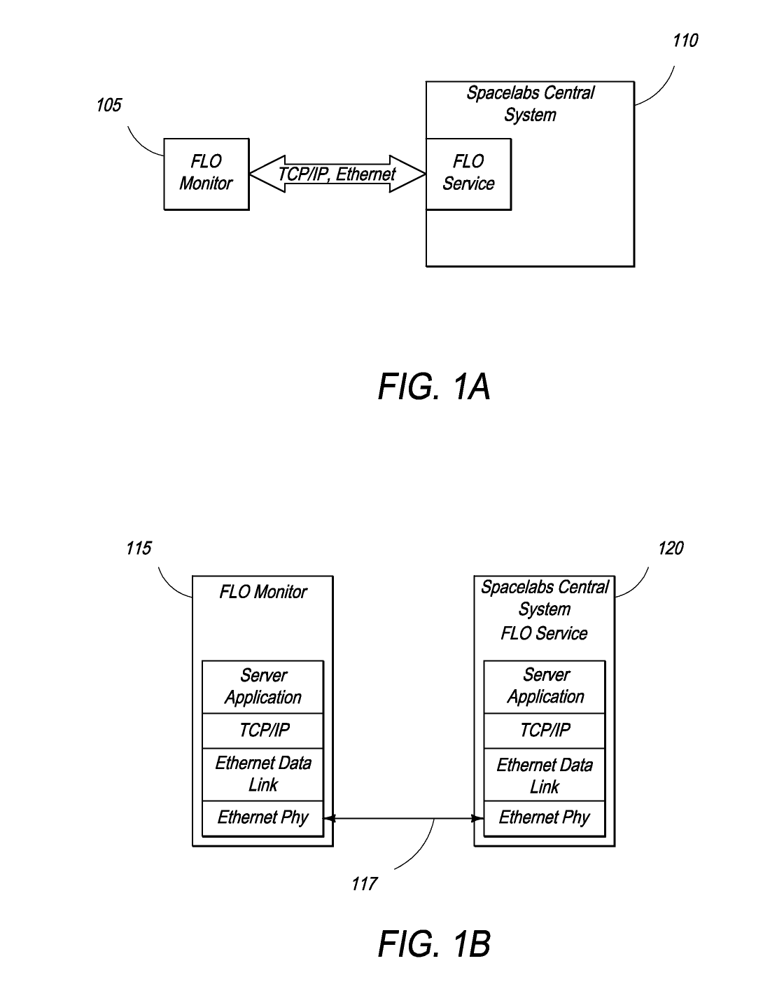

FIGS. 1A and 1B are diagrams of an exemplary communications interface between the patient monitoring system of the present invention and a larger, centralized patient database. As shown in FIG. 1A, the interface allows the patient monitoring system 105 of the present invention to acquire patient physiological data and send that data to the central system 110 if connected to the network. In one embodiment, the interface uses the TCP/IP suite protocol.

The relationship between the protocol stacks in shown in FIG. 1B. Thus, the patient monitoring system of the present invention 115 is comprised of programmatic modules, including a client application, TCP/IP stack, Ethernet data link, and Ethernet physical layer, that acts as a DNS client. The communication 117 is a packet push mechanism between the patient monitoring system 115 and the central database 120, including a client application, TCP/IP stack, Ethernet data link, and Ethernet physical layer, where data is pushed at specified rates. On receiving of packet the receiver shall send an acknowledgement packet.

In Spot Check mode, uploading of measurement records to the central database is through user-initiation. A Spot Check patient record consists of patient demographic as well as physiological data. The monitor acquires physiological data from multiple patients, which can be multiple sets of measurements for each patient, and stores all these measurements locally. The patient monitor sends all of these records to the central database once connection is established. Along with the measured data, in one embodiment, the patient monitoring system of the present invention also sends manually-entered data values for respiration rate, temperature, temperature measurement source like (tympanic, forehead, rectal, etc.), pain scale value, pain scale type, and any other fields, such as those shown in Table 1 below.

TABLE 1
Manually Entered Data Values

| MANUAL ENTRY FIELD | SELECTIONS | WHERE VIEWED |
|---|---|---|
| Caregiver I.D. | Text Entry Field | Monitor screen |
| Patient Position (during NIBP | Sitting; Supine; Standing | Not viewed on monitor |
| measurement) | | screen |
| Temperature Measurement Site | Oral; Axillary; Manual | In CIS/HIS system only |
| Manual Temperature Value (if not | XXX.X° F.; XX.X° C. | Monitor screen; Monitor |
| measured by the monitor) | | trend views; Spacelabs |
| | | applications; CIS/HIS |
| | | system |
| Pain Scale Used | Wong-Baker Faces | In CIS/HIS only |
| | (WBF); FPS-R; FLACC; | |
| Pain Score | Generally 1-10; | Spacelabs Clinical Access; |
| | FLACC (0-45) | CIS/HIS system |
| Glucose Value | XXX mg/dL; mmol/l | Spacelabs Clinical Access; |
| | (Canada & Europe) | CIS/HIS system |
| Respiration Rate | XX bpm | Spacelabs Clinical Access; |
| | | CIS/HIS system |
| WNL (Within Normal Limits) [Can be | Yes; No | In CIS/HIS only |
| used for Post-Surgical Incision | | |
| Inspection, General Assessment, etc.] | | |

In Continuous Monitoring mode, the patient monitoring system of the present invention will push numeric data at 15-second intervals when connected to the central system. The patient monitor sends collected waveform data at 1-second intervals when connected to the central system. The monitor will only send patient data while the monitor is connected to the network.

In one embodiment, the patient monitoring system of the present invention is capable of storing and displaying multiple physiologic measurement sets per patient for multiple patients. In Continuous Monitoring mode, the monitor is capable of storing and displaying a minimum of 72 hours of patient data viewable down to one-minute resolution. In Spot-Check mode, the monitor is capable of storing a minimum of 12 hours of physiologic data per patient for a minimum of 1000 patients.

In one embodiment, the patient monitor of the present invention is capable of interfacing with an optional, external 2-channel recorder that is a) designed for use in transport and b) designed to quickly connect and disconnect to/from the monitor. In one embodiment, the recorder is used to print physiologic measurements via direct connection. In another embodiment, an optional printer is employed to print physiologic measurements, such as a laser printer via a network connection. In one embodiment, the user can simultaneously print physiologic data and transfer that data to an HIS/CIS.

In Spot Check mode, the patient monitoring system of the present invention is capable of printing a single patient record. The monitor, in one embodiment, is capable of printing all patient records in a single user-initiated sequence. In Continuous Monitoring mode, the patient monitoring system of the present invention is capable of printing the SpO₂ waveform with associated vitals (SpO₂, NIBP, Temp, PR). In instances where no SpO₂ waveform is present, the strip recording is printed in the same format as the single Spot Check record format. All recordings include, but are not limited to, patient demographic data per the user-defined Patient Identification settings for recordings and the Patient I.D. display zone.

In one embodiment, the monitor retains all stored data and configuration settings should inadvertent power loss be experienced, such as through manual shutdown by a user, depletion of battery power in the absence of AC/DC power, etc. Further, the user can transfer stored patient data to an HIS/CIS. If switched between operating modes, the monitor will retain all previously stored patient data and the data is available for viewing when returned to its previous operating mode.

In one embodiment, the patient monitoring system of the present invention also includes alarm settings, supporting three levels of alarm notification, including High/Lethal, Medium and Low/Technical.

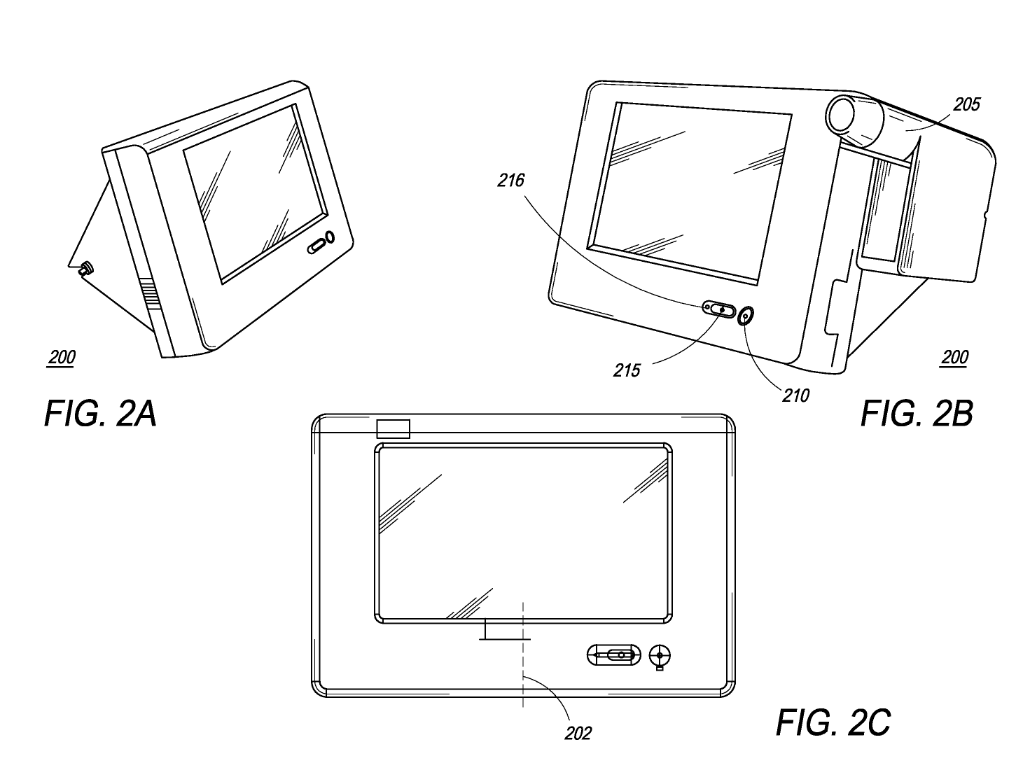

FIGS. 2A and 2B illustrate different perspective views of one embodiment of the portable patient monitoring system of the present invention. In one embodiment, the portable patient monitoring system 200 includes an integrated, 7.x-inch widescreen, touch enabled display 202, as shown in expanded view in FIG. 2C. The portable patient monitoring system of the present invention further includes a temperature sensor or probe holder 205, an AC connection indicator LED 210, an on/off button 215 and associated LED power indicator 216.

The patient monitor 200 is preferably lightweight and, in one embodiment, weighs approximately 4.0 pounds. The patient monitor 200 is also capable of standing upright when placed on a flat, horizontal surface such as a countertop, and is not easily tipped over forward or backward. Further, the patient monitor is capable of being mounted for easy and secured installation and quick release to facilitate patient transport as needed. In one embodiment, a common rear mounting design shall be adhered to (i. e. VESA) for all monitor mounts, including Wall Mounts, Roll Stand Mounts and Bedrail Hook. In another embodiment, the monitor is capable of being mounted to a wall mount or roll stand without requiring removal of a bedrail hook mount should one be attached.

In one embodiment, the patient monitor 200 features an integrated, rugged handle to facilitate portability of monitoring. The monitor is, in one embodiment, small, lightweight and readily converted to/from stationary and portable applications and is simple and easy to use. In another embodiment, the patient monitor of the present invention is ruggedized in that it is shielded from liquid splash and liquid ingress, thus minimizing cross-contamination and is primarily usable within the hospital environment.

In one embodiment, the patient monitor of the present invention is capable of operating on AC and battery power. In one embodiment, the patient monitor does not require an external power supply for AC power operation. In one embodiment, the monitor can be run on a lithium ion battery. In one embodiment, the battery is not removable by the user. In one embodiment, the operating time on the battery is a minimum of (5) hours with NIBP measurements every 15 minutes. In addition, when connected to AC mains power, the monitor automatically recharges the battery while maintaining operation. In one embodiment, an independent LED 210 is provided for ease of viewing battery charging and AC power status.

In one embodiment, the patient monitor of the present invention has at least two USB ports (not shown) for connection of peripheral devices such as barcode scanner (i.e. omni-directional with support for scanning IBST, 128, and 2D), keyboard (preferably medical grade with integrated touchpad control), mouse, USB storage/memory drive (to support software/firmware updates), and any other peripheral that may be used with the present invention.

In another embodiment, the patient monitor of the present invention is further equipped with both oral/axillary temperature probes and rectal temperature probes. Oral/axillary probes may be distinguished from rectal probes by the use of different colors on the probe and well kit, such as blue for oral/axillary and red for rectal. Further, the patient monitor of the present invention supports use of all temperature sensors/probes via a single port connection style (¼″ stereo plug) and is compatible with 400-series temperature supplies. The probes are preferably stored in temperature probe holder 205.

In one embodiment, the patient monitor of the present invention is equipped with a single NIBP hose connection port (single connection, quick release) for all supported patient types, including adult and pediatric. The patient monitoring system of the present invention supports the use of the following Spacelabs' TruLink hoses: Single Tube, Adult, 9 ft (275 cm) and Single Tube, Adult, 12 ft (366 cm). The monitor also supports the use of Spacelabs TruLink single tube NIBP cuffs (Reusable and Single Patient Use versions).

Further, the patient monitor supports the use of the following single tube, reusable, and/or anti-microbial treated cuffs:

| PATIENT TYPE | ARM CIRCUMFERENCE |
|---|---|
| Child | 5.1-7.9 in. (13-20 cm) |
| Small Adult | 7.1-10.2 in. (18-26 cm) |
| Adult | 10.2-13.8 in. (26-35 cm) |
| Adult Long | 11.4-15.0 in. (29-38 cm) |
| Large Adult | 12.6-16.5 in. (32-42 cm) |
| Large Adult | 13.8-17.3 in. (35-44 cm) |
| Thigh | 16.5-19.7 in. (42-50 cm) |

In one embodiment, a graphical user interface (GUI) is presented on the display that can adapt to different workflows, allowing caregivers to document, with ease and accuracy, vital signs measurements or initiate bedside monitoring. The patient monitoring system of the present invention, in one embodiment, displays a dynamic window GUI, since the displayed content can be varied based on patient, clinician need, and selected Monitor Profile, as previously described. In one embodiment, the patient monitoring system (with the exception of the power on/off button) is controlled via a touchscreen interface or USB-connected peripheral device. In one embodiment, the touchscreen GUI of the present invention is used to enable a user to switch operating mode between continuous monitoring and spot check.

In one embodiment, the GUI can be used to adjust factory default settings such as, but not limited to patient type; high/low alarm limits (continuous monitoring mode), high-low limit violations (spot check mode); range limit violations (spot check mode); alarm message priorities (high, medium and low); NIBP rest period (between readings in Auto mode); and NIBP initial inflation pressure. In one embodiment, in order to adjust default settings, the user must enter a password.

In one embodiment, the GUI is employed to enable/disable the display of units of measure (UoM) such as mmHg/kPa, ° C./° F., etc. In one embodiment, the GUI employs offsetting colors, which can be user-defined, to help differentiate between the various clinical data measurements, labels, icons, etc. In addition, the GUI may display unique visual indicators to differentiate between measurements. For example, “above the range” may be indicated as “++++ ”, below the range may be indicated as “−−−− ” and unreadable may be indicated as

In one embodiment, the patient monitoring system of the present invention is capable of supporting display of trended data and measurements in two formats: mini-view and window view. In mini view, the display on the patient monitoring system will show a subset of all available data elements in a sub-zone of the monitor's “Normal Screen” display. The clinician can change display formats to show trend data/measurement records specific to one patient (by Patient I.D.) or multiple patients (chronologic listing of completed readings). In window view, the clinician is able to initiate display of a larger tabular trend window for viewing the full list of supported trend data elements. Display of the window view hides the mini view, NIBP and temperature parameter numeric zones.

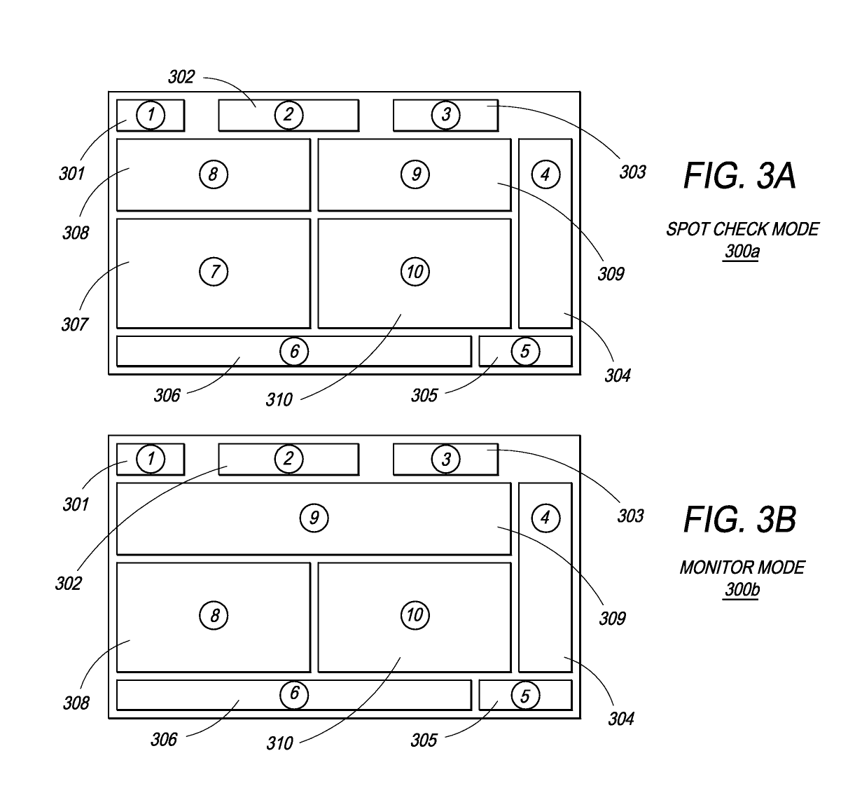

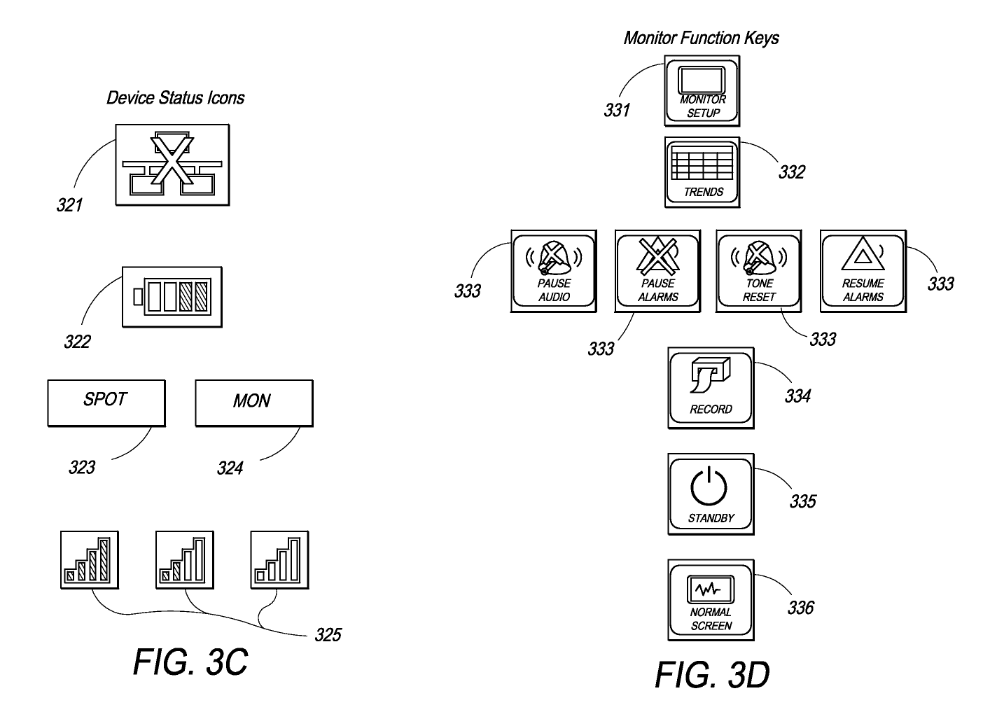

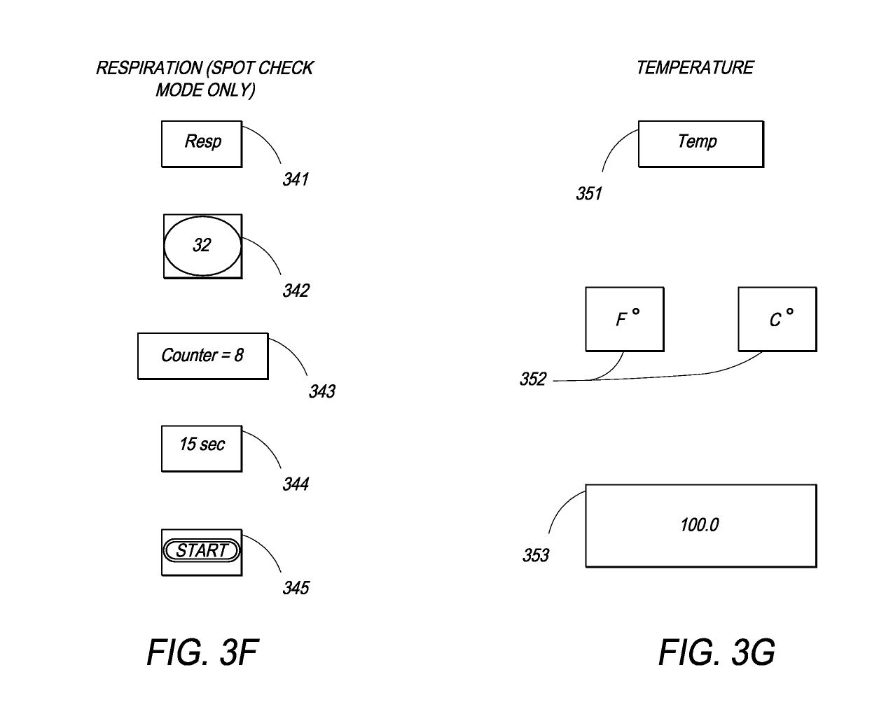

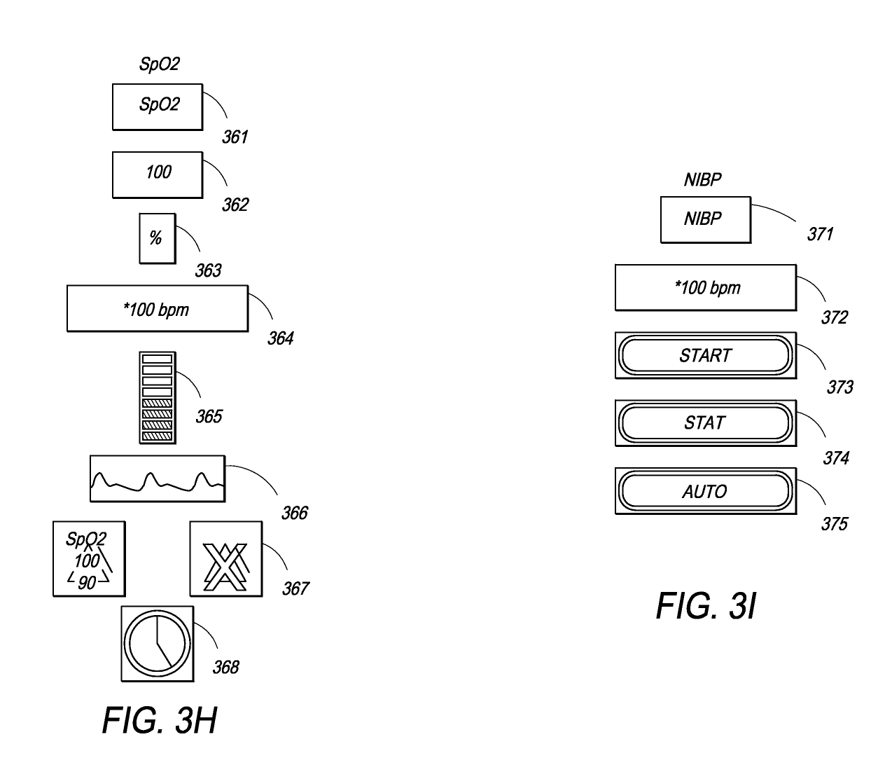

As described in greater detail below in FIGS. 3A and 3B with respect to each operating mode, the screen appearance between each operating mode differs and/or the screen may be annotated with a mode indicator such that it is obvious to the users which mode the monitor is configured for. Also, as mentioned above, the GUI is a dynamic window GUI, since the displayed content can be varied based on patient and clinician need. Further, when the operating mode is changed, the patient monitor produces an on-screen confirmation prompt, with the user being required to acknowledge the change prior to the operating mode actually changing.

FIGS. 3A and 3B represent illustrative views of general exemplary layouts of GUIs 300a and 300b in both Spot Check mode and Continuous Monitoring mode, respectively. More specifically, FIG. 3A and 3B represent “normal” screen views of the patient monitoring system of the present invention. In one embodiment, the GUI provides icon-based menus to provide quick access to frequently used features. Further, measurements are displayed in large, easy to read numerics. Referring now to FIGS. 3A and 3B, simultaneously, area 301 is a device identification area, where the identification of the device will appear. In one embodiment, the device ID is alpha-numeric.

Area 302, in one embodiment, is a patient identification area for presenting patient identification information, in a numeric, alpha, or alphanumeric format. It should be noted that the patient demographics that can be viewed in this area can be user-defined. If patient demographics are manually entered, a patient demographic screen can be accessed by touching area 302, which acts as a hot-key. When area 302 is touched again, the demographic screen will disappear.

Area 303, in one embodiment, is the device status/information area. The device status information area is for displaying a plurality of device status icons, as shown in FIG. 3C. Device status icons include, but are not limited to LAN icon 321 for displaying wired network status; battery icon 322 for displaying the battery power meter; operating mode icons 323, 324 for indicating whether the patient monitoring system of the present invention is in spot check mode or monitoring mode, respectively; and WiFi icon(s) 325 for displaying wireless network status and signal strength.

Area 304, in one embodiment, is employed to provide monitor function key icons, as shown in greater detail in FIG. 3D. As shown in FIG. 3D, monitor function icons include, but are not limited to monitor set-up icon 331, which causes the monitor set-up menu to open when touched. In addition, trend button 332, is provided for accessing a “Normal Trends” view. Further, alarm controls 333 may be provided, including, but not limited to tone reset and pause audio, which is used to silence the audio alarm for a period, such as 15, 30, 45, 60, 90, or more seconds; alarm suspend or pause alarm, which is used to suspend audio alarms for a period, such as 1, 2, 3, 4, 5, or more minutes; and resume alarms, which cancels the pause alarm condition. The use of alarm control keys 333 triggers a message in message area 305. Further, monitor function keys include a record key 334 for initiating recording for a predetermined period of time, where the recording destination is a peripheral or back-end device, such as a printer. Standby key 335 is provided, which in Spot Check mode, dims the system and puts it in power saving mode and, in continuous monitoring mode, suspends processing. Normal screen key 336 is provided to allow the clinician to revert back to a “home” state screen configuration where no menus are open.

Referring back to FIGS. 3A and 3B, area 305 is used for displaying date/time. Date may be presented in a plurality of formats and can be adjusted by an administrator. Time, in one embodiment, is displayed in military 24 hour clock format (HH:MM:SS), thus saving space in views because the AM/PM indicator is not needed.

Area 306 is employed as a message bar to alert the clinician to a plurality of messages, such as alarm or battery status. It may also be used to indicate disconnection of a patient sensor or of a back-end device or peripheral, such as a printer.

Area 307 is reserved for the respiration measurement area, when operating in Spot Check mode only. Referring now to FIG. 3F, respiration measurement area 307 includes a parameter label 341, an indication of the units of measure, a respiration value and tap pad area 342, where the respiration value is only displayed within the tap pad after the tap duration has completed. A user must accept the value if it is acceptable, otherwise, redo the respiration measurement. Respiration tap counter 343 is provided to display the number of taps that have been registered in the tap duration. The counter update in real time in conjunction with screen taps. Tap duration setting 344 is also provided so that the user can select the tap interval duration of 15/30/60 seconds. Start key 345 is provided to initiate the tap count. In addition, a redo key may be provided to re-initiate a tap count, a value adjustment key may be provided to adjust the displayed respiration count or to facilitate entry of the respiration rate, and an accept key may be provided so that the displayed value is accepted and logged into the system.

Referring now to FIGS. 3A and 3B, simultaneously, area 308 is used as the temperature zone. Details of the icons in the temperature zone are illustrated in FIG. 3G. Referring to FIG. 3G, parameter label 351 is provided to indicate that this area is reserved for temperature measurement and indicators. The unit of measure 352 is also displayed to aid the clinician and is user adjustable. The temperature value indicator 353 is provided to show the temperature value and is user adjustable. In addition, an alarm status indicator may be provided, as may an indicator of temperature mode (oral, axillary, manual, etc.). Further, when configured for Spot Check mode, the system presents the user with a “Reading in Progress” indication, when the patient temperature is being measured. A time stamp indicator may also be provided to indicate when the last temperature reading was completed.

Area 309 is employed as the SpO₂ zone, in one embodiment. Details of the icons in the SpO₂ zone are illustrated in FIG. 3H. Referring to FIG. 3H, parameter label 361 is used for indicating that it is the SpO₂ measurement zone. SpO₂ value indicator 362 and unit of measure 363 are also provided in area 309. The HR/PR value 364 is displayed only if the SpO₂ channel is active. If displayed, it will display continuously. Signal quality Indicator 365 is provided as a dynamic graphic icon wherein the color bars fill up the icon relative to signal quality. In addition, SpO₂ waveform 366 is provided. The alarm status indicator 367 is provided to indicate the status of the SpO₂ measurement. A time stamp may be provided to indicate when the SpO₂ reading was completed. In addition, indicators specific to particular type of oximeter sensors or circuit boards may be used or provided.

Area 310 is employed as an NIBP zone, in one embodiment. Details of the icons in the NIBP zone are illustrated in FIG. 3E. Referring to FIG. 3E, parameter label 371 is provided to indicate that the zone is for NIBP measurement. A portion of area 310 is used to display the NIBP value as systolic/diastolic or mean. Heart rate/pulse rate value 372 is available when the SpO₂ channel is inactive. In addition, the indication of a unit of measure may be provided in this area. Further, an alarm status indicator may be provided in this area. In addition, measurement mode keys, such as keys 373, 374, 375, may be provided, where the monitor of the present invention can display up to four of these mode keys, which can be specified by the Administrator.

The primary distinctions between the normal screen views in Spot Check and Continuous Monitoring mode include: a) in the SpO₂ zone area 309, in continuous monitoring mode, a continuous SpO₂ waveform is displayed across the width of the screen, while in Spot Check mode, no SpO₂ waveform is displayed and b) in the monitor function keys/icon area 304, the selection and arrangement of these keys may vary between operating modes; for example, in Spot Check mode, the alarm tones key, or any alarm related icons, are not needed, due to the lack of alarms in this mode.

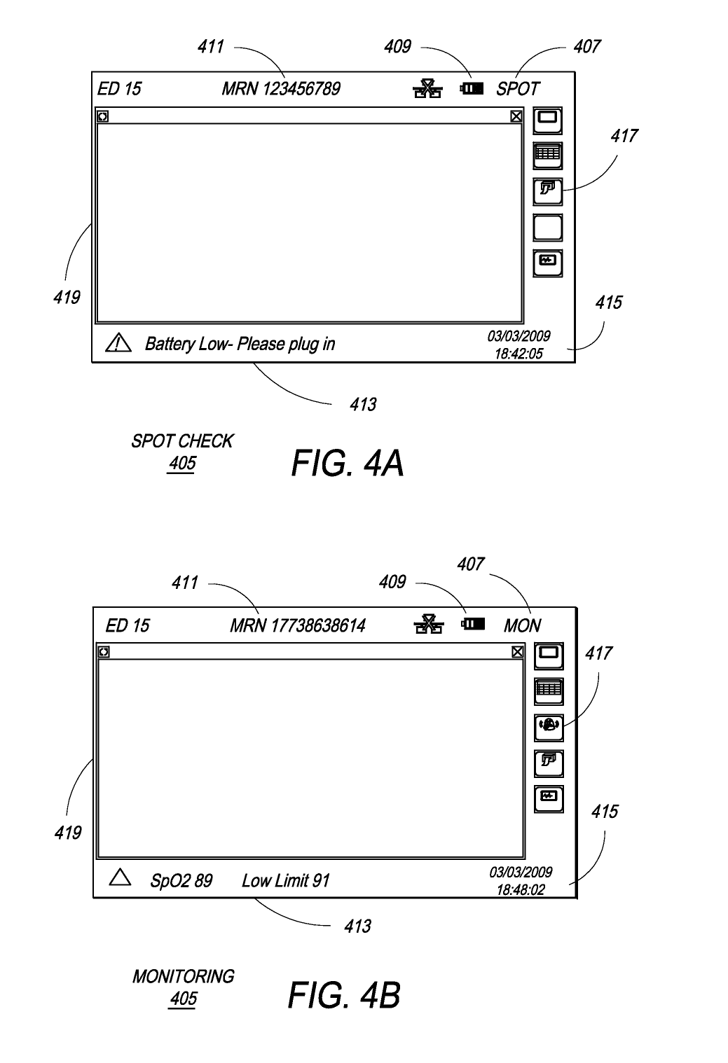

The patient monitoring system of the present invention displays a dynamic window GUI, since the displayed content can be varied based on patient and clinician need. Two exemplary GUIs are shown in FIGS. 4A and 4B, showing an application window view for Spot Check mode and Continuous Monitoring mode, respectively.

Referring now to both FIGS. 4A and 4B, the application window view GUI 405 includes a mode indicator area 407, a battery level indicator area 409, patient ID indicator 411, a status area 413, time and date area 415, menu icons 417, and parameter zone area 419. As shown in FIG. 4A, when the monitoring system is in Spot Check mode, menu icons 417 may include “monitor setup”, “trends”, “record”, “save”, and “normal screen”. As shown in FIG. 4B, when the monitoring system is in Continuous Monitoring mode, menu icons 417 may include ““monitor setup”, “trends”, “pause audio”, “record”, and “normal screen”. Further, when in spot check mode, mode indicator area 407 reads “SPOT”, while when in continuous monitoring mode, mode indicator area 407 reads “MON”. In one embodiment, when the display switches from Spot Check mode to Continuous mode, the “save” button is removed, grayed out or otherwise deactivated and the “pause audio” button is added or activated. In one embodiment, when the display switches from Continuous mode to Spot Check mode to, the “pause audio” button is removed, grayed out or otherwise deactivated and the “save” button is added or activated. This change, caused by transitioning from Spot Check mode to Continuous mode or vice-versa, can be applied to any button, icon, or displayed data variable.

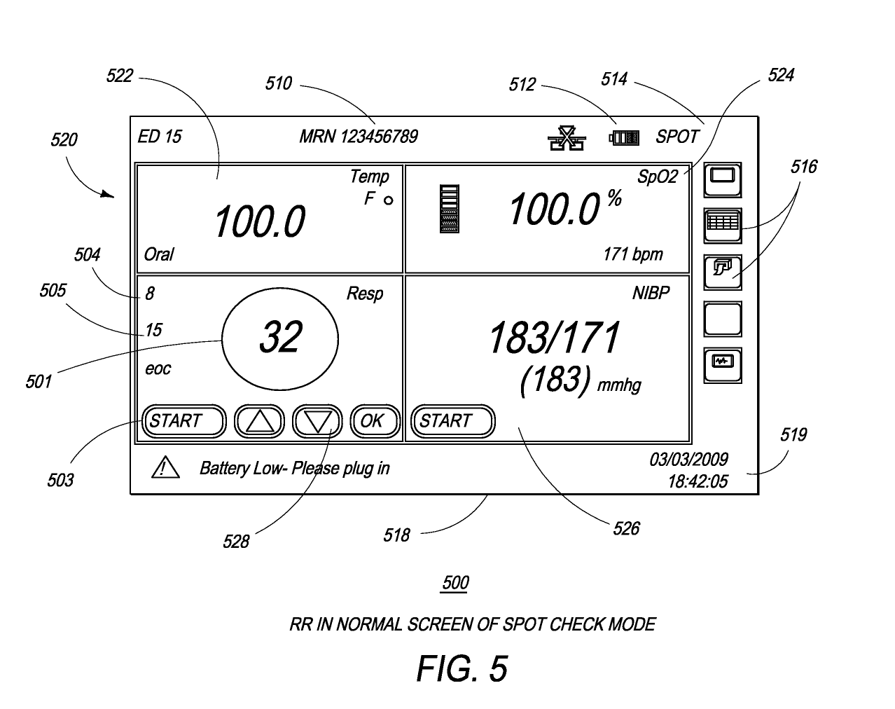

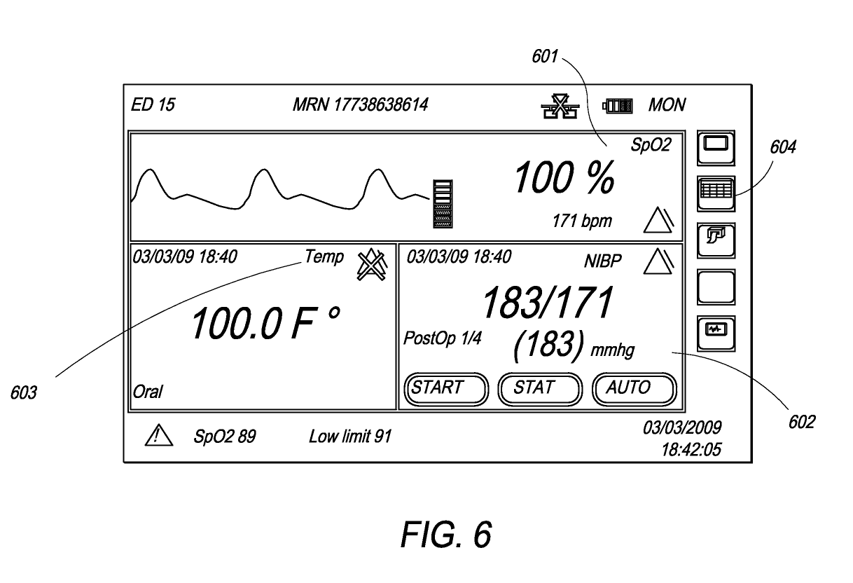

When opened, the application window occupies all parameter zone areas, as shown in FIG. 5 for Spot Check mode and FIG. 6 for Continuous Monitoring Mode.

As shown in FIG. 5, an exemplary application window for Spot Check mode includes patient ID area 510, battery level indicator 512, mode indicator 514, menu icons 516, status area 518, time/date area 519 and parameter zone area 520. Parameter zone area 520 further includes temperature status indicator area 522, SpO2 display area 524, NIBP display area 526, and respiration rate (RR) area 528.

In one embodiment, the patient monitoring system of the present invention provides for a facile method for manual measurement and entry of respiration rate, shown as area 528 in FIG. 5. Current patient monitoring products support measurement and display of respiration rate (RR) in one of two ways. First, in monitoring products that include either ECG or EtCO₂, RR is typically measured by one of those two parameters. However, in low-acuity type products (vital sign monitors, spot-check monitors, etc.) that do not include ECG or EtCO₂ RR is typically a value measured manually by the caregiver (via stethoscope) and manually-entered into the low-acuity monitor or directly documented in a charting system.

The process of the caregiver manually measuring the patient's RR requires the caregiver to apply a stethoscope to the patient's chest or back. The caregiver then listens for complete respirations, counting them over a period of time to arrive at a RR/minute value. As one example, the caregiver may count respirations for 15 seconds and then multiply that by (4) and then subtract “1” to calculate a “per minute” RR. Upon completion, the caregiver would then manually input the patient's RR into the low-acuity monitor either via physical input device (i.e. keyboard, numeric keypad) or via the monitor's user interface (UI) (i.e. on-screen keypad or scroll arrows).

The patient monitoring system of the present invention includes a mechanism for facilitating manual measurement of the RR by the caregiver, with automated entry of the RR value into the low-acuity monitor without further intervention by the caregiver. In one embodiment, the mechanism is a graphical user interface (GUI) 500, which is depicted to the user in the form of a window 501 on the monitor's screen, as shown in FIG. 5. The GUI window 501 acts as a “touch pad”, whereby the user taps the window concurrent with each respiration the caregiver detects through the stethoscope. This allows the caregiver to focus on listening for the respirations and can help automate the process for manually measuring the RR. It should be appreciated that, while the display itself can be used as the input, other input devices can be used, including a mouse pad, touchpad, keyboard key, or other input device capable receiving a repeated tap from a user.

In operation, the caregiver first manually activates the “touch pad” that is part of the GUI 500 by touching Start button 503. Once activated and displayed on-screen, the systems' counting mechanism starts at a count of “1” beginning with the first screen tap by the caregiver, which is input based upon a manually detected physiological signal, and is displayed in count area 504. In one embodiment, the device employs a mechanism to notify the user when their tap is accepted, which optionally includes one or more of an alert sound, a flashing light, or a vibration. Additionally, at the first screen tap, the system begins auto-counting a predetermined, configurable time period (such as 15-seconds), where the time period is displayed in area 505. At the completion of 15 seconds, the monitor automatically calculates the RR as [(Number of Screen Taps*4)−1]. Optionally, the tap collection may continue for an indefinite period of time and stop when the user hits a stop button available on the device GUI. Referring to FIG. 8, the process is depicted as a) the user initiating 801 the tap pad by touching a start icon, or some other indicator, b) the user initiating 803 a physiological monitoring (such as listening for a respiration or a pulse), c) the user tapping 805 into the input device in relation to the manually detected physiological parameter (i.e. one tap for each detected respiration or one tap for each detected pulse), and d) the user completing 807 the measurement (because a predefined time period has expired or because the user has indicated the tap measurement should terminate, described in greater detail below). Once the measurement has completed, the system automatically calculates the desired physiological parameter.

The “touch pad” then disappears and the calculated value is automatically entered into the corresponding “RR” field of the monitor screen. At this point, the RR value has been entered in as part of the patient record which can then be sent on to an HIS/CIS system. In one embodiment, the device is activated when the user starts tapping the screen and calculates a running average of all the breath to breath intervals (converted into breaths per minute) units. It should be appreciated that the touch pad and screen tap mechanism can be used for measuring not only the respiration rate, but also for measuring other physiological parameters, such as but not limited to pulse rate.

Further, since the tap counter is touch activated and measures the number of taps in a given length of time, the rate of taps can be used to determine the rate of several kinds of events in a medical context. For example, the tap rate counter may be used for counting the number of occurrences of a particular type of behavior that a clinician is monitoring. Thus for instance, if a speech therapist is working with a patient who stammers, and wants to determine how frequently the patient struggles with a certain sound, they can tap at each instance of the behavior and get an estimate of the rate of the event and a count of the number of times it occurred. By eliminating the need for an observer to mentally track each occurrence of an observed physiological event and allowing the observer to simply translate an observed physiological event into a repeated physical action (tapping, pressing, clicking, or some other simple motor motion), the tap pad allows a caregiver to focus on monitoring the physiological event. Such physiological events can include respiration, pulses, speech impairments, twitches, SpO₂ data, ECG data, or other data.

Accordingly a user can use the tap pad for any interval or rate measurement at the bedside, including for respiration rate, pulse rate, or ECG.

In one embodiment, the time period is fixed. In one embodiment, the fixed time period is system-defined. In another embodiment, the fixed time period is user-defined. In another embodiment, the time period may be selected by a user from a menu of pre-defined choices. In another embodiment, the time period is variable and is dynamically determined by the system when the user starts and stops the input, such as tapping.

In one embodiment, the user can run the tap pad for a fixed period of time, such as 5, 10, 15, 20, or 30 or more seconds, whereby the data is collected by the device. The data collected by the device represents the time of each physical input (taps, clicks, touches, etc) by the user. The physical input data is recorded and processed by the device, which subsequently produces a numeric average. Thus, the device generates a plurality of different computations and displays, including calculating an average rate using all data. The device also displays the current average, in real time, based on the interval data collected to date. For example, after two physical inputs, the user has defined an interval which can be used to calculate the respiration rate of the patient in breaths per minute. This value is displayed on the screen. When a third physical input is recorded, the two defined intervals are averaged and the display is updated. Thus, the display is continuously updated with each physical input by the user. Note that the display update can include any applicable filtering chosen to be applied by the user (for example, remove longest and shortest interval or select a median).

In one embodiment, the device uses the average of the interval data to determine the value of the physiological parameter. In one embodiment, the method for calculating the average interval data is user-configurable. In one embodiment, the average interval data is derived by calculating a straight arithmetic average of the measured intervals. In another embodiment, the average interval data is derived by first rejecting the longest and shortest interval data (outliers) generated by the physical inputs and then averaging the remaining data. In yet another embodiment, the average interval data is derived by calculating a median value of the physical inputs.

In yet another embodiment, the average interval data is derived by analyzing clusters of intervals and using a clustering algorithm to reject long or short intervals that should not be included. Optionally, the user is presented with a histogram of the interval set collected, displaying the intervals used in the rate calculation, allowing the user to choose from the histogram which intervals are included and excluded in the calculation and, in cooperation therewith, displaying the intervals that are being included and excluded and allowing the user to edit those intervals by clicking or touching the appropriate bars on the histogram, and displaying the results in any unit defined by the user, including breaths per minute, beats per minute, etc.

It should be appreciated that the display system can comprise a menu which can be used to define the specific variables used to define the operation of the tap pad, including a) time period for running a tap pad measurement and b) type of data being gathered (respiration rate, pulse rate, etc.), which, based on the particular applied algorithm, will govern the calculation and display of any final values in accordance with the type of data gathered.

In one exemplary usage scenario of the patient monitoring system of the present invention, the system is used in Spot Check mode by a clinician, such as a CNA. The clinician will take vital signs readings from multiple patients and at the completion of rounds, will connect the device to a network for transfer of data to the central patient database.

In the following usage scenario, it should be assumed that the clinician has a list of patients that need to be monitored, the patient may or may not have existing records, there is no alarm set, and the system is not connected wirelessly. In use, the clinician captures both the Patient ID along with patient data, as described in detail above. Then, the clinician applies the appropriate sensors to the patient for obtaining measurements. The clinician then verifies settings of the system and subsequently initiates readings, such as NIBP and temperature. Then, the clinician verifies the readings and confirms the Patient ID. The clinician can also enter modifiers, including his or her own user ID. In addition, the clinician can save and/or print, delete, or even retry the measurements. The clinician can then move on to the next patient.

Once all measurements are taken the clinician can batch send the results of all patients to the network, verify that the data has been transferred, and change record status for sent records or delete. Uploading of Spot Check measurement records to the database is effectuated via user-initiation. The monitor will then provide local confirmation of successful uploading of patient records. The monitor will annotate all records uploaded to the database with a “sent” indication after confirmation of successful uploading. The monitor will also provide local indication of inability to upload patient records. In the event patient records are not uploadable, the monitor will prompt the user to re-verify the Patient I.D. before uploading can be re-attempted.

In addition, a visual alert is provided to denote whether a connection to the database has been achieved. The monitor provides an indication of patient records that have not successfully been sent to the central system. For patient records that have not successfully been sent to the central system, the monitor allows for the user to initiate one of the following actions: a)

MODIFY & RE-SEND: the user can modify and re-send the unsent records. (This implies the user has verified the Patient I.D. and corrected it); b) ACCEPT AS NEW: the user can re-send the unsent records, instructing the central system to create a new patient record based on the present Patient I.D. and any other demographic data specified (i.e. Patient Name); or c) DELETE: the user can delete the unsent records from the monitor's database, which will also remove those records from the trend table view.

The system may alert to the following error conditions, including battery low, switch between modes, failure to send or connect, printer out of paper, device lock-up, hardware failure, or improperly entered patient identification (more commonly, “fat finger Patient ID”). Improperly entered patient identification refers to a state that occurs when a user enters a bad patient ID (SSN, MRN, birth date etc.) at the touch screen. The error state indicates that a bad patient id has been entered and requires a fix by the user to get the data associated with the correct patient.

In another embodiment, the patient monitoring system of the present invention can be operated in Continuous Monitoring mode (CMM). CMM has local alarms and programmable episodic measurement capture. In CMM, the patient monitoring system of the present invention performs real-time, continuous monitoring of physiologic status and support local alarm notifications. Referring to FIG. 6, the system window, in one embodiment, displays measurements of SpO₂ 601, NIBP 602, and non-invasive temperature 603. In addition, the user is able to manage the ongoing collection and storage of measured vitals for a patient through the initiation of a Start/Stop process. Other window parameters have been described above with respect to FIG. 5 and will not be repeated herein. Rather, the distinctions between Spot Check Mode and Continuous Monitoring Mode are highlighted.

In an exemplary usage scenario of the patient monitoring system of the present invention used in Continuous Monitoring Mode, the clinician, such as a CNA is a primary user. In the following usage case scenario, it should be understood that the device is dedicated to one patient, a network is present, and there is no central surveillance. In use, the clinician associates the patient ID with an initial data set, if available, via a network connection.

The clinician then applies the appropriate sensors to the patient for measurement of parameters. Then, the clinician selects a patient profile to populate with automatic settings. Next, the settings are clinician-verified, including alarm settings. Initial modifiers may be loaded by the clinician as well. The clinician then initiates the collection of first readings of data and monitors local alarms. The system begins capturing vital signs, such as SpO2, pulse values, NIBP, and temperature, in addition to alarm events and setting limits. It is also possible to view trends (using the icon 604 shown in FIG. 6) with alarms in various modes, such as tabular at auto-mode frequency or tabular at an alternate rate or full data set.

The monitor is capable of monitoring a patient and trending vitals without first requiring any patient demographic data to be entered. If no patient demographic data has been entered, and patient monitoring has been initiated, the monitor will automatically enter an auto-assigned Patient I.D. into the corresponding demographic field.

In cases where no signal or measurements are detected/recorded for one minute or longer, upon re-detection of signal(s), the monitor will prompt the user to confirm whether the monitor is still connected to the same Patient. If NIBP and/or temperature are the only parameter(s) being monitored, then the monitor's prompt (for the user to confirm whether the monitor is still connected to the same Patient) will occur at the time the next NIBP and/or temperature measurement is attempted.

If the user confirms the monitor is still connected to the same patient, the monitor will continue to write and store monitored data to the currently entered Patient I.D. If the user indicates the monitor is not connected to the same patient, the monitor will prompt the user to indicate whether the existing patient record should be purged and a new patient record created (per Auto Patient I.D. assignment). If the user does NOT indicate the existing patient record should be purged, then the patient record will remain in the monitor, and all subsequent monitored data will be stored to that patient record. The monitor will send settings information to the central system when connected. When finished monitoring any particular patient, the patient is disconnected from the device and data is purged from the device upon confirmation from the clinician.

The system may alert to the following error conditions, including battery low, switch between modes, failure to send or connect, printer out of paper, device lock-up, hardware failure, or fat finger Patient ID, as described above with reference to Spot-Check mode.

In yet another embodiment, the patient monitoring system of the present invention can be operated in a NIBP Protocol Mode, as shown in FIG. 7. The NIBP protocol mode allows the user to create a custom schedule for taking automatic NIBP readings. The NIBP protocol mode may be applied in continuous monitoring mode. While existing monitoring equipment allow the caregiver to take BP measurements at a fixed schedule (such as once an hour, or once every 15 minutes), the NIBP protocol mode of the present device allows for the creation of a measurement schedule that can be variable. Thus, for example, when a patient is being monitored immediately after arrival into the intensive care unit, initially the clinician sets up a protocol for measurement of BP at every 15 minutes, for a period of 2 hours. At the same time, the clinician may also configure the system to take measurements at 30 minute intervals for 4 hours (after the initial 2 hours have elapsed), and then at 1 hour intervals until 10:00 pm, followed by every 2 hours from 10:00 pm until 6:00 am.

As shown in FIG. 7, in NIBP protocol mode, the GUI screen 700 provides buttons 701 that may be used by the clinician to setup and edit customized measurement schedules. In this manner, the device provides the capability to create many protocol schedule profiles that can be selected by caregivers to suit the needs of individual patients.

Various changes could be made in the above constructions without departing from the scope of the invention, therefore, it is intended that all matter contained in the above description should be interpreted as illustrative and not as limiting.

We claim:

1. A system for determining a physiological parameter of a patient, the system comprising:
an input device for receiving a plurality of physical inputs from a user and communicating to a memory data indicative of the frequency at which said plurality of physical inputs are received from the user within a time period; and
a processor in data communication with said memory wherein said processor determines the value of a physiological parameter based upon said data.

2. The system of claim 1, wherein said data collected by the device represents the time of each physical input by said user.

3. The system of claim 1 wherein said physiological parameter is respiratory rate.

4. The system of claim 1 wherein said physiological parameter is at least one of pulse rate or respiratory rate.

5. The system of claim 1 wherein the input device is a touch screen display.

6. The system of claim 5 wherein the plurality of physical inputs received from the user are finger-taps on said touch screen display.

7. The system of claim 1 wherein the input device is at least one of a touch screen display, mouse pad, or keyboard.

8. The system of claim 7 wherein the plurality of physical inputs received from the user are finger-taps on at least one of said touch screen display, mouse pad, or keyboard.

9. The system of claim 1 wherein the time period is fixed.

10. The system of claim 9 wherein the fixed time period is system-defined.

11. The system of claim 9 wherein the fixed time period is user-defined.

12. The system of claim 1 wherein the time period is variable and is dynamically determined by the system when the user starts and stops the physical input.

13. The system of claim 1 wherein the processor determines the value of the physiological parameter by averaging all of interval data generated by said physical inputs.

14. The system of claim 13 wherein the average interval data is derived by calculating a straight arithmetic average of the measured intervals.

15. The system of claim 13 wherein the average interval data is derived by rejecting the longest and shortest interval data generated by the physical inputs and then averaging the remaining data.

16. The system of claim 13 wherein the average interval data is derived by calculating a median value of the physical inputs.

17. The system of claim 13 wherein the average interval data is derived by analyzing clusters of intervals and using a clustering algorithm to reject long or short intervals that should not be included.

18. The system of claim 13 wherein the average interval data is displayed in real time and continuously updated with each physical input.

19. A system for determining a physiological parameter of a patient, the system comprising:
an input device for receiving a plurality of physical inputs from a user, wherein said physical inputs comprise finger taps, and communicating to a memory data indicative of the frequency at which said plurality of physical inputs are received from the user within a time period, wherein said data comprises interval data; and
a processor in data communication with said memory wherein said processor determines at least one of the patient's respiration rate or pulse rate based upon said data.

20. The system of claim 18 wherein the processor determines the value of the physiological parameter by averaging all of interval data generated by said physical inputs.
